(19) United States
(12) Patent Application Publication
(10) Pub. No.: US 2026/0018275 A1
(43) Pub. Date: Jan. 15, 2026

(54) Controlling or Regulating a Medical Scanner in a Scanner Fleet

(71) Applicant: Siemens Healthineers AG, Forchheim (DE)

(72) Inventors: Rainer Schneider, Höchstadt (DE); David Grodzki, Erlangen (DE); Tobias Würfl, Erlangen (DE); Mario Zeller, Erlangen (DE); Thomas Möck, Nürnberg (DE); Armin Stranjak, Uttenreuth (DE)

(73) Assignee: Siemens Healthineers AG, Forchheim (DE)

(21) Appl. No.: 19/266,243

(22) Filed: Jul. 11, 2025

(30) Foreign Application Priority Data
Jul. 12, 2024 (DE) 10 2024 206 594.3

(51) Int. Cl. G16H 30/20; A61B 5/00; G06F 3/04847

(52) U.S. Cl. CPC G16H 30/20; A61B 5/7475; A61B 2560/0209; G06F 3/04847

(57) ABSTRACT

A computer-implemented method for controlling or regulating an imaging medical device in a fleet of imaging medical devices of a predetermined modality. The computer-implemented method includes accessing at least one digital database in order to receive measurement metadata with respect to at least one image acquisition or with respect to the imaging medical device. The image acquisition was performed using the imaging medical device. The received measurement metadata is analyzed using a large language model (LLM) with respect to at least one technical parameter of the image acquisition and/or of the imaging medical device. A program code is determined based on the analysis of the at least one technical parameter. The program code is determined for controlling or regulating the imaging medical device. The determined program code is provided.

### TECHNICAL FIELD

A technique for controlling or regulating an imaging medical device in a fleet of imaging medical devices of a predetermined modality is provided. The technique comprises, in particular, a method, a computing unit, a system, a computer, program product, and a computer-readable storage medium.

### BACKGROUND

In the course of the consolidation of hospital chains, it is considered a priority in radiology to operate and maintain a large number of MRT scanners across distributed sites. At the same time, it is more and more difficult to recruit qualified personnel for operating the MRT scanners, with the result that in some cases rotating or remotely curated (remote) working models are established.

The standardization and homogenization of the MRT fleet of the large radiology houses/chains is therefore becoming increasingly important. In this regard, detecting deviations, increasing efficiency and conducting analyses of the different MRT devices, sometimes with specialization on specific organ regions (‘neuro’ vs ‘cardio’ scanners), is a highly complex and iterative process.

Fleet analysis is currently supported by centrally provided ‘dashboards’ with graphic visualizations in relation to different issues. Currently, there are two sources for dashboard generation. On the one hand, direct analyses take place by means of machine log data. Current states and developer debugging information are written in log data onto the scanner. A subgroup of this log information is extracted centrally and analyzed. On the other hand, implicit analyses are conducted by means of DICOM tags. By interpretation of DICOM tags it is implicitly possible to make inferences on a scanner-specific basis in relation to activity and measurement throughput and other details. The images are, in most cases, captured centrally by a PACS system for analysis, which, of course, is reduced to the information contained in the available DICOM images (not all are sent to the PACS system) and their tags (the tags only contain certain information). A common feature of both methods is that it is necessary to interpret and graphically edit textual information.

In this case, the provided means of editing and the issues are fixed, and therefore not always appropriate for the questions facing the specific fleet and problem analyses of deviations, and/or do not ideally reflect the way of thinking of the person conducting the analysis.

In practice, it is also often difficult to find the chain of the right dashboards. As a result of the knowledge gained from a visualization, there often follows a follow-up question, for which a different dashboard must again first be found, which renders the process inefficient.

Furthermore, newly introduced log information and/or DICOM tags cannot be interpreted directly, and analysis algorithms must constantly be updated.

### SUMMARY

It is therefore an object of the present disclosure to provide a solution for an improved control or regulation of the scanners of a scanner fleet of a predetermined modality. Alternatively, or in addition, the object consists of optimizing image quality, energy consumption, and/or utilization of the capacity of the scanners within the fleet. Further, alternatively or in addition, the object consists in enabling further improvements in the control and regulation in the light of future changes to the scanner fleet hardware and/or scanner fleet software.

This object is achieved by means of a method for controlling or regulating an imaging medical device in a fleet of imaging medical devices of a predetermined modality, by means of a computing unit, by means of a system, by means of a computer program (and/or a computer program product) and by means of a computer-readable storage medium according to the attached independent claims. Advantageous aspects, features, and aspects are described together with advantages in the dependent claims and in the following description.

The solution, according to the disclosure in relation to the claimed method as well as in relation to the claimed computing unit, is described in the following. Features, advantages, or alternative aspects herein can be attributed to the other claimed subject matters (e.g., the system, the computer program, or a computer program product) and vice versa. In other words, the claims for the computing unit and the system comprising the computing unit may be improved by features that are described or claimed in connection with the method and vice versa. In this case, the functional features of the method are embodied by means of structural units of the computing unit or the system and vice versa.

According to one aspect of the method, a (in particular computer-implemented) method for controlling or regulating an imaging medical device in a fleet of imaging medical devices of a predetermined modality is provided. The method comprises a step of accessing at least one digital database in order to receive measurement metadata in respect of at least one image acquisition or in respect of the imaging medical device. The image acquisition was performed by means of the imaging medical device. The method further comprises the step of analyzing, by means of a large language model (LLM), the received measurement metadata with respect to at least one technical parameter of the image acquisition and/or of the imaging medical device. The method further comprises the step of determining a program code based on the analysis of the at least one technical parameter. The program code is determined for controlling or regulating the imaging medical device. The method further comprises the step of providing the determined program code.

By means of the technique according to the disclosure, an image acquisition by means of imaging medical devices of a predetermined modality of a medical institution or a network of medical institutions is analyzed interactively, dynamically, extendably, iteratively, user-specifically, and/or “on the fly” and, if necessary, modified. The analyzing and, if necessary, modifying (e.g., by means of program code for controlling or regulating) can be conducted with high precision and specificity in respect of a medical issue. In the context of the technique according to the disclosure, “on the fly” can signify that a permanent or temporary storage of data in a permanent data memory is dispensed with. For example, the measurement metadata can be analyzed directly (e.g., without long-term storage on a computing unit performing the method or in a cloud performing the method). Alternatively, or in addition, the provided program code does not need to be stored over the long term. For example, a result of the analysis of the measurement metadata can be deleted within a predetermined period of time after being provided as program code.

The fleet of imaging medical devices (also: scanner fleet) of a predetermined modality can comprise all the scanners of the modality (e.g., all MRT scanners) of a medical institution or a network of medical institutions (also: fleet operators; in short: operators). For example, the scanner fleet can have a shared planning system and/or shared (in particular trained) operating personnel.

The at least one digital database can comprise a picture archiving and communication system (technical acronym: PACS) and/or an electronic health record system (for short: eGA, German acronym for electronic health record; also: electronic patient record, ePA; technical acronym: electronic health record, EHR; also: electronic medical record, EMR; electronic patient record, ePA). Alternatively, or in addition, the at least one digital database can comprise a local database of the medical imaging device (also: scanner), for example a log database.

The measurement metadata can be metadata in respect of the image acquisition (also: acquisition and/or measurement) and in particular comprise metadata of acquired image data, for example, as a DICOM header (DICOM, standing for Digital Imaging and Communications in Medicine). The DICOM header may, for example, contain a medical issue. Alternatively, or in addition, the measurement metadata can be metadata in respect of the medical imaging device (e.g., the MRT scanner). For example, the measurement metadata can comprise machine log data and/or measurement metadata of the imaging device. The machine log data and/or measurement metadata can comprise technical data (for example, technical parameters) before, during, and/or after image acquisition or the acquisition of the image data. For example, the machine log data and/or measurement metadata may contain data indicating idle times (also: dead times) of the scanner and/or times when the scanner is in an energy-saving mode. Alternatively, or in addition, the machine log data and/or measurement metadata may comprise data concerning completed image acquisitions whose image data was not selected for storing in the PACS. Alternatively or in addition, the measurement metadata may be metadata of the imaging medical device (for short also: of the device or scanner) relating to device support, e.g., power supply (such as, say, the number of power outages), network connection (e.g., bandwidth fluctuations, etc.). Alternatively, or in addition, the measurement metadata of a number of devices in the fleet may be aggregated in order to enable an analysis to be conducted and/or process variables and/or control variables for the fleet to be calculated.

The accessing of the at least one digital database may comprise an accessing of two or more digital databases, for example accessing a PACS and the log databases of one or more (in particular all) scanners within the fleet.

The two or more digital databases may contain different types of measurement metadata. For example, the PACS may comprise selected acquired image data containing DICOM headers, and the log databases of the scanners by means of which the image data was acquired may comprise machine log data and/or other measurement metadata.

The LLM (also: large language model) can be designed and/or trained to analyze and/or semantically combine the measurement metadata from at least one (in particular from the different) digital database(s), in particular with respect to at least one technical parameter. The technical parameter may be necessary for answering a question in relation to the fleet of imaging medical devices. For example, the technical parameter may comprise the utilization of capacity, the operating time, the dead time, the number of examinations, the number of protocols used, and/or the type of protocols, and/or other key indicators.

The LLM can represent a computer-linguistic probability model that has learned statistical word and sentence string relationships from a multiplicity of text documents (in particular comprising DICOM header, machine log data, and other measurement metadata and/or comprising an initial program code) as a result of a computationally intensive training process.

The program code can control or regulate an energy consumption (e.g., a transition between acquisition mode and energy-saving mode) and/or an acquisition protocol. The program code can be scanner-specific (or device-specific), acquisition-protocol-specific, or universal for a plurality of (in particular, all) scanners of the fleet.

The provision of the program code may comprise an outputting of a control signal or regulation signal. Alternatively, or in addition, providing the program code may comprise an output of the program code at a user interface (UI), for example, a graphical output on a graphical user interface (also: user interface or GUI). The output at the user interface can comprise a graphic (also: dashboard) or some other visualization of historical and/or statistical data. The data output can relate to a single scanner or to a number of scanners (all scanners in the fleet, for example).

The method can be performed in fulfillment of data protection requirements. For example, the at least one digital database can be arranged within a computer system of the medical institution or the network of medical institutions and/or only be accessible within a local network (e.g., a local area network, LAN). Alternatively, or in addition, the data of the at least one digital database can be access-protected and/or encrypted.

The method can be performed by a (in particular central) computing unit of the scanner fleet. The (in particular central) computing unit may be an “edge device”. The “edge device” may be integrated in an internal computer system of the medical institution and/or be provided as a cloud system for the network (fleet of imaging medical devices) via a network connection, e.g., the internet.

The method can be performed locally, in a distributed manner, and/or in a (e.g., local) cloud.

The predetermined modality may be or comprise magnetic resonance tomography (MRT), computed tomography (CT), positron emission tomography (PET), single-photon emission computed tomography (SPECT), X-ray, and/or a hybrid method of the aforementioned modalities, in particular PET/CT or PET/MRT.

The scanner fleet may be an MRT scanner fleet, for example. For example, a capacity utilization, an energy consumption, and/or image qualities of the MRT scanner fleet (or any other scanner fleet, e.g., a CT scanner fleet) can be optimized by means of the technique according to the disclosure.

The program code may comprise a machine code, in particular in order to control or regulate the energy consumption and/or the use of at least one acquisition protocol.

The machine code can be determined for changing at least one (e.g., at least one analyzed) technical parameter of the scanner or of a plurality of scanners within the fleet (e.g., all scanners). The technical parameter may be, for example, a parameter that differentiates between an energy-saving mode and an acquisition mode, in particular, which activates the energy-saving mode or configures its activation (e.g., with respect to time and/or on an event-driven basis).

By means of the machine code, it is possible to switch between different states of the scanner, for example, between the energy-saving mode and the acquisition mode. Alternatively, or in addition, the machine code may be used in order to differentiate between an image acquisition according to a first acquisition protocol and an image acquisition according to a second acquisition protocol, and in particular to control, in particular limit, the availability of the acquisition protocols on the scanner.

The first and second acquisition protocol may be different from one another for example in terms of a duration of the image acquisition, an overall energy consumption, a radiation dose and/or a measurement sequence (e.g., in MRT).

The provision may comprise an outputting at a user interface (UI). The method may further comprise a step of receiving a user input via the user interface (UI) in respect of the provided program code.

The method may further comprise a step of modifying the determined program code based on the received user input. The method may further comprise a step of providing the modified program code.

The outputting of the provided program code at the user interface (UI) may comprise a “prompt”.

The “prompt” may, on the one hand, be a request to a user (also: operator) on the UI to actuate a user input (for short: input). On the other hand, the term “prompt” may also be understood as the respective user input in response to the request.

The prompt or the user input may be used for controlling the LLM.

The user input may comprise a response message (also: feedback and/or assessment) in respect of the provided program code.

A targeted modification (e.g., an optimization) of the program code can be realized as a result.

The method may further comprise a step of checking the determined or modified program code with respect to at least one consistency condition. The step of providing the program code can be performed when a result of the check confirms that the at least one consistency condition has been met.

By checking at least one consistency condition, it can be demonstrated that the provided program code is functionally fit for purpose, technically correct and/or reliable, and/or that the program code satisfies official requirements (technical term: compliance).

The at least one consistency condition may comprise an executability of a machine code, a compiler cross-check, a syntax analysis, a technical check, a reliability, an adherence to specified regulations and guidelines and/or a validity.

In the context of the technique according to the disclosure, a compiler is a computer program that translates source code of a particular programming language into a form that can be executed (e.g., more directly) by a computing device or a computer. By successfully completing a compiler cross-check, it is possible to guarantee a (e.g., more or less directly) executable program code.

The syntax analysis can ensure that the program code is functionally fit for purpose and useful or target oriented.

By observing the specified regulations and guidelines (also: compliance) it is possible to achieve a conformity with statutory, regulatory and/or company inhouse standards.

The technical checking and validation of the program code can ensure a technical executability in each case.

The method may comprise a step of receiving a user input via a user interface (UI) in order to determine the program code based on the received user input.

A user input can be received before the step of analyzing the measurement metadata by means of the LLM (for example in order to conduct the analysis in a targeted manner for an actual issue), before the determining of the program code (for example in order to weight an analysis result), and/or after providing the program code (for example in order to modify the program code).

The method may comprise a step of post-training the LLM by means of the received measurement metadata and/or by means of the received user input.

The post-training of the LLM can be accomplished on the basis of newly received measurement metadata. In some exemplary aspects, the post-training of the LLM may further be conducted based on one or more received user inputs, for example on the basis of the preselection of the criteria and/or on the basis of the feedback in relation to the provided program code. The feedback can be received in the form of a user input.

The receiving of the user input may comprise a preselection of criteria (e.g., in respect of scanners, acquisition protocols and/or anatomical regions of a patient group) on the basis of which the LLM analyzes the received measurement metadata. For example, the user input may comprise a medical issue for which the image acquisition is to be performed.

The user input may be received in response to a prompt (also: user invitation).

The user interface (UI) may be embodied as a graphical user interface (GUI) and/or comprise a microphone.

The provision of the determined (and/or modified) program code may comprise a graphical visualization by means of the GUI.

The program code may be provided as one or more dashboards. A dashboard (e.g., in information management) can refer to a graphical user interface that serves for visualizing data. The program code may specify how and when data is output on the UI (e.g., the form of the visualization, the overlaying of visualizations, and/or the selection and/or combination of datasets, etc.).

The analysis of the at least one technical parameter and/or the analysis of measurement metadata of the imaging medical device (for example of one or more MRT scanners) can be output in an intuitively easily understandable form by means of the GUI and/or the one or more dashboards.

The provision of the determined or modified program code may comprise an outputting of a control signal or regulation signal. Optionally, the control signal or regulation signal may be domain-specific, in particular device-type-specific, parameter- and/or software-specific, and/or acquisition-protocol-specific.

By means of the control signal or the regulation signal, it is possible to modify at least one technical parameter for image acquisitions of one or more (in particular, all) scanners from the fleet. For example, an idle time can be shortened, and/or an acquisition protocol modified or replaced.

The control signal or regulation signal can be determined for activating an energy-saving mode, for modifying (in particular shortening) an idle time up to the activation of the energy-saving mode, for modifying or replacing an acquisition protocol of at least one imaging medical device and/or for modifying time scheduling plans for image acquisitions (in particular for a predetermined anatomical region of a patient group).

The control signal or regulation signal for activating the energy-saving mode (also: standby mode) may comprise a predetermined duration for an inactive time (also: dead time; technical term: idle time) following an image acquisition. After the predetermined period of time, the energy-saving mode can be activated by means of the control signal or regulation signal. The control signal or regulation signal can, for example, shorten a preset time period in order to achieve an overall energy saving.

Alternatively, or in addition, there may be no preset time period default for a duration of an idle time up to the activation of the energy-saving mode.

Further alternatively or in addition, the energy-saving mode may be activatable as a function of a time of day and/or a weekday. For example, regularly occurring idle times can be determined on the basis of the analysis of the measurement metadata and an activation of the energy-saving mode at the regularly occurring idle times can be determined. Typically, for example, a scanner can be regularly in use Monday to Friday during the day. At night and at weekends, the scanner may be deployed only in unscheduled emergency situations such that an overall energy saving can be achieved by activation of the energy-saving mode of the scanner at night and at the weekend provided no unplanned emergency situation has occurred. Following an unscheduled deployment, the scanner can then be switched once again into the energy-saving mode at night and at the weekend after a short idle time.

Modifying or replacing the acquisition protocol may, for example, comprise a shortening or an energy-saving adjustment of the acquisition protocol. By shortening the acquisition protocol, a greater number of image acquisitions can be achieved by means of the scanner. Replacing the acquisition protocol may comprise transferring an acquisition protocol from one scanner to another scanner. The transfer of the acquisition protocol can be accomplished via the cloud, the (in particular central) computing unit, and/or a different edge device.

Modifying the time scheduling plans for image acquisitions may comprise optimizing (in particular shortening) intervals between the start of sequential image acquisitions for a predetermined type of image data.

The anatomical region of a patient group may for example comprise image data of a knee region (e.g., in the case of a suspected torn ligament) and/or image data of a thorax region (e.g., in the case of a suspected heart attack, some other suspected cardiac and/or vascular disease, and/or a suspected lung infection), and/or image data of a head region (e.g., in the case of a suspected brain tumor or stroke).

The patient group may comprise patients with a similar suspected diagnosis, a similar examination objective, and/or a similar treatment objective.

The modification or replacement of the acquisition protocol and/or the acquisition of images of the predetermined anatomical region of the patient group may comprise an administration of contrast agent (in particular an omission of an administration of contrast agent in the interest of time optimization) and/or a deployment (and/or omission and/or replacement) of items of equipment (for example measurement coils specific to the anatomical region, such as e.g., knee coils). For example, an acquisition protocol can be simplified and/or shortened by performing just one image acquisition instead of two image acquisitions that are to be compared with each other, one without and one with contrast agent. Alternatively, or in addition, an acquisition protocol can be simplified and/or shortened by, for example, dispensing with a multichannel coil of an MRT system.

The at least one digital database may comprise at least one vector database and/or an embedded database system.

A vector database can be a database system that serves for storing and searching for vectors. The database system can be optimized for an efficient search for similar vectors within the database. The vectors can be high-dimensional representations of unstructured data, such as the image acquisitions and/or describing texts. A cosine similarity and/or a Euclidean distance can be used as a similarity metric for the vectors. The most similar vectors can be identified, for example, by means of predefined threshold values (technical term: thresholding; for example, comprising a maximum of N most similar vectors for a natural number N) and/or by weighting the N best vectors. Alternatively, or in addition, an approximate nearest neighbor search can be used.

An embedded database system can be a database system which is embedded in an application software package and does not visibly appear externally. An embedded database system must not be identifiable as such from the outside and/or cannot be usable for data storage by external systems. The advantages of embedded database systems result from the fact that the vendor can perform an adjustment aligned to the specific application (in particular, the medical image acquisitions of the predetermined modality), which goes beyond the possibilities of normal administration and acceleration. At the same time, data protection guidelines can be complied with.

A further advantage is easier installation and licensing of a product (in particular of a scanner and/or its software) that uses an embedded database system. The product manufacturer is able to ship the product to customers as a whole. Licenses for the product can be negotiated between the product manufacturer and the customer without the involvement of the database vendor. The product manufacturer can conclude a licensing agreement with the database vendor without the involvement of customers.

The at least one digital database and/or the LLM can use one or more predetermined database languages. For example, SQL (technical term also: Structured Query Language) can comprise a database language for defining data structures in relational databases as well as for processing (for example, for inserting, modifying, and/or deleting) and querying data resources based thereon.

A semantic search and/or semantic analysis of the received measurement metadata are/is made possible by means of the vector databases of the embedded database system and/or of the predetermined database languages.

The analysis may comprise a semantic attribution and/or structured consolidation of the measurement metadata.

The at least one technical parameter may comprise an energy consumption of an imaging medical device per unit time, a number of image acquisitions by an imaging medical device per unit time, a number of image acquisitions per unit time as a function of an acquisition protocol and/or a number of image acquisitions per unit time as a function of an acquired anatomical region of a patient group.

In one exemplary aspect, an energy efficiency of one or more scanners within the fleet is analyzed by means of the LLM. An energy consumption over a predetermined period of time (also: determined per unit time) is determined on the basis of the received measurement metadata. An average energy consumption (for example, for a predetermined acquisition protocol and/or a predetermined medical issue) over all scanners within the fleet can be determined, along with deviations of the scanner or of a number of scanners. It can be determined whether and/or when an energy-saving mode is active on the scanner or should be active. A length of an idle time before the switchover into the energy-saving mode can be checked and optimized (e.g., shortened).

In a further exemplary aspect, a scan efficiency can be analyzed. For example, a number of image acquisitions, a number of patients, and/or a number of measurements over a predetermined period of time (also: per unit time) can be determined. An average value can be determined, for example, as a function of the anatomical region (also: per body region), and deviations of individual scanners can be determined. An acquisition protocol and/or a number of measurement steps can be optimized. For example, an acquisition protocol measurement time can be shortened by reducing the number of measurement steps (e.g., no separate measurements without and with contrast agent) and/or by modifying a parameterization of the measurement steps.

In a further exemplary aspect, a scanner capacity utilization can be analyzed. For example, a timeline and/or variations with respect to time during low utilization of the scanner can be analyzed. In particular, the analysis can take the acquired anatomical regions (also: measured body regions) into consideration.

The exemplary aspects can be combinable with one another in order to optimize a capacity utilization of the scanner fleet and/or an overall energy efficiency of the scanner fleet and/or to reduce a time delay of image acquisitions.

The analysis of the at least one technical parameter may be scanner-specific, specific to a type of activity (e.g., which anatomical body regions are typically acquired by means of a scanner), specific to an acquisition protocol and/or specific to a type of measurement (also: image acquisition).

By means of the analysis of the at least one technical parameter, a program code can be determined in order e.g., to activate an energy-saving mode. This can be accomplished e.g., via a programming interface (technical term: application programming interface, API). For example, a Boolean flag can be set in a service software layer of the scanner. Alternatively, or in addition, an idle time up to the switchover into the energy-saving mode can be adjusted, in particular for the entire scanner fleet. For example, an e.g., integer value can be set and/or changed in the control software (e.g., as an idle time in ms) in order e.g., to transfer a gradient amplifier (GPA) for example of an MRT scanner into the energy-saving mode (also: standby mode) after the idle time has elapsed.

The modification or replacement of the acquisition protocol may comprise replacing a measurement program by the measurement program of another scanner. In an exemplary aspect, the measurement program can be transferred from scanner B (or the other scanner) via the (e.g., central) computing unit, the edge device, the cloud and/or a network drive to scanner A (the scanner). Alternatively, or in addition, a push update can be performed on the scanner by a central protocol management server. The acquisition protocol or measurement program can be modified or replaced via an HTTP REST interface of the scanner.

In the case of an acquired anatomical region (also: body region) with many idle times (also: dead times), a utilization of capacity can be optimized by a change to the time scheduling.

The method can be performed on a central computing unit, on an edge device, and/or in a cloud.

In an exemplary aspect, the method can be performed on an edge device or some other central computing unit of the medical institution or of the network of medical institutions. In a further exemplary aspect, the method can be performed distributed over a plurality of hardware units, for example, in a local cloud of the medical institution or of the network of medical institutions. Alternatively, or in addition, the edge device may be embodied as hardware that connects the local network to the external internet.

According to a device aspect, a computing unit is provided for controlling or regulating an imaging medical device in a fleet of imaging medical devices of a predetermined modality. The computing unit comprises a database interface which is designed for accessing at least one digital database in order to receive measurement metadata in respect of at least one image acquisition or in respect of the imaging medical device. In this case, the image acquisition was performed by means of the imaging medical device. The computing unit further comprises an analysis module which is designed to analyze, by means of an LLM, the received measurement metadata in respect of at least one technical parameter of the image acquisition and/or of the imaging medical device. The computing unit further comprises a program code determination module, which is designed to determine a program code based on the analysis of the at least one technical parameter. The program code is determined for controlling or regulating the imaging medical device or for the analysis of measurement metadata of the imaging medical device. The computing unit further comprises a program code interface or a program code provider module, which is designed to provide the determined program code.

The computing unit may be a central computing unit and/or an edge device.

The computing unit can be designed to perform the method according to the method aspect. Alternatively, or in addition, the computing unit can comprise any feature that is disclosed in the context of the method according to the method aspect.

According to a system aspect, a system is provided for controlling or regulating an imaging medical device or for analyzing measurement metadata of an imaging medical device in a fleet of imaging medical devices of a predetermined modality. The system comprises a computing unit according to the immediately preceding claim. The system further comprises at least one digital database in which measurement metadata, in particular in respect of at least one image acquisition that was performed by means of the imaging medical device, is stored.

The system can be designed to perform the method according to the method aspect. Alternatively, or in addition, the system can comprise any feature that is disclosed in the context of the method according to the method aspect.

According to a further aspect, a computer program product is provided comprising program elements which cause a computing unit to perform the steps of the method for controlling or regulating an imaging medical device in a fleet of imaging medical devices of a predetermined modality according to the method aspect when the program elements are loaded into a memory of the computing unit.

According to yet a further aspect, a computer-readable medium is provided on which program elements are stored which can be read and executed by a computing unit in order to perform steps of the method for controlling or regulating an imaging medical device in a fleet of imaging medical devices of a predetermined modality according to the method aspect when the program elements are executed by the computing unit.

### BRIEF DESCRIPTION OF THE DRAWINGS

The above-described characteristics, features, and advantages of the present disclosure, as well as the manner in which they are achieved, will become clearer and more easily understandable in the light of the following description and the exemplary aspects, which are explained in more detail in connection with the drawings. This following description does not limit the disclosure to the included aspects. Like components or parts may be labeled with the same reference signs in different figures. Generally, the figures are not to scale.

It goes without saying that a preferred aspect of the present disclosure may also comprise any combination of the dependent claims or of the above aspects with the respective independent claim.

These and other aspects of the disclosure will become apparent from the below-described aspects and are explained with reference to these.

FIG. 1 is a flowchart of a method for controlling or regulating an imaging medical device in a fleet of imaging medical devices of a predetermined modality according to a preferred aspect of the present disclosure.

FIG. 2 is an overview of the structure and the configuration of a computing unit for controlling or regulating an imaging medical device in a fleet of imaging medical devices of a predetermined modality according to a preferred aspect of the present disclosure; and

FIG. 3 shows a schematic exemplary aspect of the method of FIG. 1 comprising a check on consistency conditions or “reasoning” as well as visualizations of the program code.

Any reference signs in the claims are not to be understood as limiting of the field of application.

### DETAILED DESCRIPTION

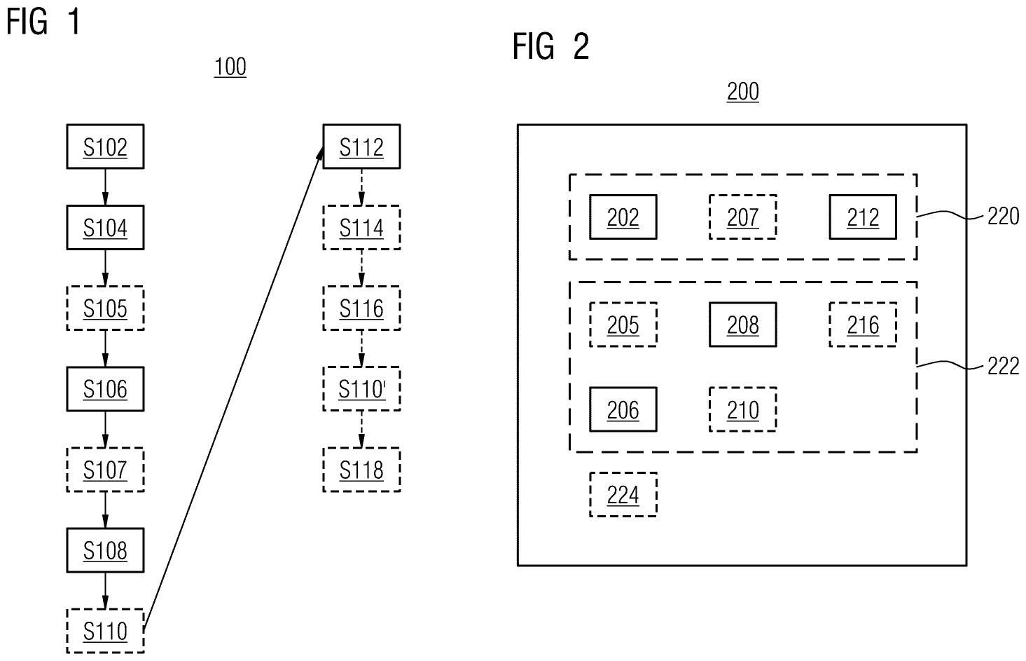

FIG. 1 schematically shows an example of a flowchart of a computer-implemented method 100 for controlling or regulating an imaging medical device in a fleet of imaging medical devices of a predetermined modality.

The method 100 comprises a step S102 of accessing at least one digital database in order to receive S104 measurement metadata in respect of at least one image acquisition or in respect of the imaging medical device. The image acquisition was performed by means of the imaging medical device.

The method 100 further comprises a step S106 of analyzing, by means of a large language model (LLM), the received S104 measurement metadata in respect of at least one technical parameter of the image acquisition and/or of the imaging medical device.

The method 100 further comprises a step S108 of determining a program code based on the analysis S106 of the at least one technical parameter. The program code is determined for controlling or regulating the imaging medical device.

The method 100 further comprises a step S112 of providing the determined S108 program code.

The method 100 may comprise a step S105 of post-training the LLM by means of the received measurement metadata and/or by means of a received (e.g., in steps S107 and/or S114) user input.

The method 100 may comprise a step S107 of receiving a user input via a user interface (UI) in order to determine S108 the program code based on the received S107 user input. The user input can be received not only at the point in the method workflow shown in FIG. 1 but also, for example, before step S105 of the post-training of the LLM and/or before step S106 of the analysis of the measurement metadata by means of the LLM.

The method 100 may comprise a step S110; S110′ of checking the determined S108 or a modified (e.g., in step S116) program code in respect of at least one consistency condition.

The method 100 may comprise a step S114 of receiving a user input via a user interface (UI) in respect of the provided S112 program code. In a step S116, the determined S108 program code based on the received S114 user input can be modified. The modified S116 program code can be provided in a step S118.

FIG. 2 schematically shows an exemplary aspect of a computing unit 200 for controlling or regulating an imaging medical device in a fleet of imaging medical devices of a predetermined modality.

The computing unit 200 comprises a database interface 202 which is embodied for accessing at least one digital database in order to receive measurement metadata in respect of at least one image acquisition or in respect of the imaging medical device. The image acquisition was performed by means of the imaging medical device.

The computing unit 200 further comprises an analysis module 206, which is designed to analyze, by means of an LLM, the received measurement metadata in respect of at least one technical parameter of the image acquisition and/or of the imaging medical device.

The computing unit 200 further comprises a program code determination module 208 which is designed for determining a program code based on the analysis of the at least one technical parameter. The program code is determined for controlling or regulating the imaging medical device or for analyzing measurement metadata of the imaging medical device.

The computing unit 200 further comprises a program code interface 212 or a program code provider module 212 which is designed to provide the determined program code.

The computing unit 200 may comprise a post-training module 205 which is designed for post-training the LLM by means of the received measurement metadata and/or by means of the received user input.

The computing unit 200 may comprise a user input receive interface 207 which is designed to receive a user input via a user interface (UI) in order to determine the program code based on the received user input.

Alternatively, or in addition, the user input receive interface 207 can be designed to receive a user input via the user interface (UI) in respect of the provided program code.

The computing unit 200 may comprise a check module 210 which is designed to check the determined or modified program code in respect of at least one consistency condition.

The computing unit 200 may comprise a program code modification module 216 which is designed for modifying the determined program code based on the received user input.

The program code interface 212 may be further designed to provide the modified program code.

The computing unit 200 may comprise an input/output interface 220. The database interface 202, the optional user input receive interface 207, and/or the program code interface 212 may be embodied by means of the input/output interface 220.

The computing unit 200 may comprise a processor 222. The optional post-training module 205, the analysis module 206, the program code determination module 208, the optional check module 210, and/or the optional program code modification module 216 may be embodied by means of the processor 222.

The computing unit 200 may comprise a memory 224. Program elements can be stored in the memory in order to perform the method 100. Alternatively, or in addition, intermediate results or final results of the steps of the method 100 or outputs of the individual modules or interfaces of the computing unit 200 can be buffered or stored.

The computing unit 200 can be designed for performing the method 100.

A system (not shown) for controlling or regulating an imaging medical device or for analysis of measurement metadata of an imaging medical device in a fleet of imaging medical devices of a predetermined modality comprises a computing unit 200 and at least one digital database in which measurement metadata, in particular in respect of at least one image acquisition that was performed by means of the imaging medical device, is stored.

The system can be designed for performing the method 100.

The technique, according to the disclosure (for example, comprising the method 100, the computing unit 200, and/or a system comprising the computing unit 200), may also be referred to as “Generative MR Fleet Analysis with LLMs”.

A fleet analysis can be significantly simplified by means of the technique according to the disclosure. The fleet analysis can be interactive, dynamic, iterative, user-specific, and/or graphical.

All data sources (e.g., logs, DICOM tags, etc.) can be efficiently analyzed with respect to an issue by means of a large language model (LLM), and the LLM can create an associated program code, for graphical visualization, for example.

In an exemplary aspect, in order to ensure the program code (for short: code) is valid, programming-language-specific and/or program-specific ‘reasoning’ elements (also: checkable consistency conditions, for example in steps S110; S110′ of the method 100) are inserted downstream thereof, which elements filter out invalid results during the inference (e.g., via compiler cross-check, for short: compiler check). For example, based on the interpretation of the current graphic, the user can initiate a follow-up query, which in turn extends and/or modifies the existing code and thereby improves the graphical visualization, inter alia. In this way, the user can refine the graphical evaluation based on the newly acquired findings in order to analyze the fleet of imaging medical devices (e.g., MRT fleet) more effectively.

According to the technique according to the disclosure, the LLM can also modify already existing dashboards, which are either provided as standard or have been created from a preceding (also: most recent) analysis. In this case, the prompt, for example, is supplemented with the already existing code and updated by an extension with respect to the specific issue.

The user can enter their question either in a provided input window (as an example of a UI, in particular a GUI) and/or by voice input (as an alternative example of a UI).

Optionally, the user can mark a determined range of existing graphical visualizations in advance in order to reduce the question to this specific range, e.g., by marking a specific scanner, body region, or subplot.

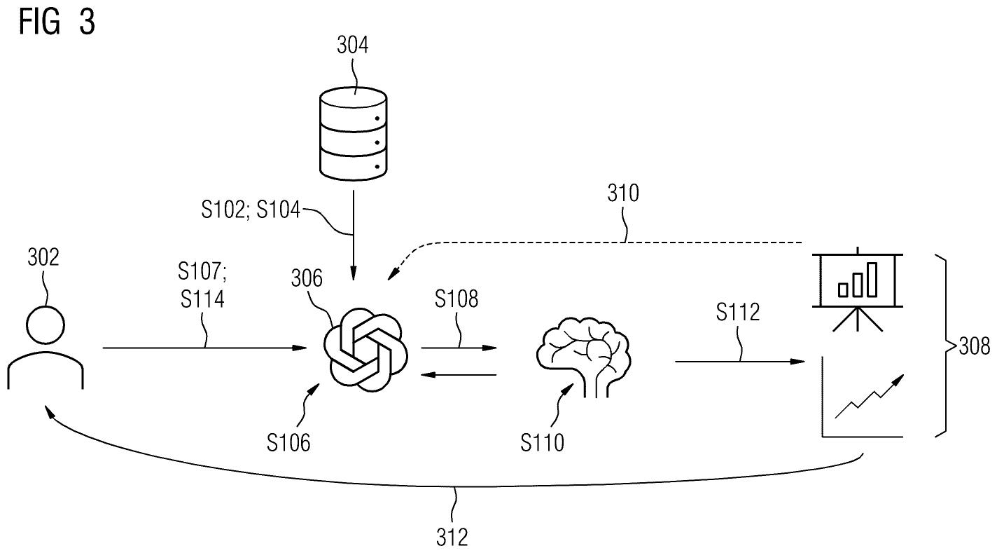

FIG. 3 shows an exemplary aspect of the technique according to the disclosure. At reference signs S107; S114, a user input is received from the user 302. The user input can, e.g., read: “Output the fleet operators or scanners that have conducted more than 3 neurological examinations in the past month”; “Display the MRT scanner with the lowest patient throughput in the past month” and/or “Determine which clinical program requires the most time on average”.

The LLM 306 performs step S106 of analyzing measurement metadata. The measurement metadata is received, as shown at reference signs S102; S104, from one or more digital databases. The measurement metadata may, for example, comprise MRT scanner logs (or machine logs, also: machine log data), DICOM tags, and/or transponder (for short: TP) receiver logs.

At reference sign S108, a program code is determined, and at reference sign S110 is checked in relation to one or more consistency conditions in a schematically depicted “reasoning” element. A compiler cross-check and/or a syntax check are/is performed, for example.

The program code provided at reference sign S112 may comprise a database language, such as SQL (e.g., SQL for Structured Query Language), for example, and/or a visual source code.

At reference sign 308, two types of graphical representations or visualizations are schematically depicted by way of example. The graphical representations or visualizations may use, for example, a program such as Qlik Sense, HTML5 Plots, and/or Power Bi for the data analysis.

At reference sign 310 in FIG. 3, an aspect is shown in which an initial source code for an existing visualization and/or prior information of elements that were marked by the user are present.

At reference sign 312 in FIG. 3, an aspect is further depicted in which the user visually interprets the graphical representations or visualizations 308. This may give rise to follow-up questions and/or a need for further analyses. For example, the user may receive a prompt or request for a user input.

Existing LLMs can be adapted and/or extended for the use case according to the disclosure in different ways that can be combined with one another.

In one aspect, a fine-tuning of the LLM can be performed. In order to optimize the LLM specifically, it can be trained further using a dataset (in particular a set of measurement metadata) that comprises user (also: operator) questions, fleet databases (e.g., comprising DICOM tags and/or log data, for example machine log data) and associated dashboard and/or visualization source codes. The data may be sourced from real datasets such that the LLM receives a better idea of which procedures (e.g., regulating or controlling actions) fit best in which contexts.

In a further aspect, the technique according to the disclosure comprises prompting. The LLM can be controlled by means of specific input requests (also: prompts) in order to deliver, inter alia, relevant and real dashboard suggestions. Over time and by way of feedback, the structure and word choice of the prompts can be refined and improved via metaprompting, which optimizes the ‘configuration’ of the LLM for this aspect (or use case). For example, a high degree of precision and/or a high specificity of the answer can be called for.

In a further aspect, the at least one digital database comprises semantic databases. The LLM can be used in combination with what is called an embedded (technical term: “embedding”) database and/or a vector database which contains user data, operator-specific data (also: customer-specific data, e.g., specific to a clinic or medical institution, and/or to a network of clinics or medical institutions, in particular with a distributed system), DICOM tags and/or dashboard or visualization source code. Such a database can enable the LLM to access a comprehensive history of cases in a semantic manner and thereby process large volumes of data in one or more prompts.

In a further aspect, a continuous learning can take place and feedback loops can be incorporated. Users can give feedback (also: user inputs) in relation to the suggestions of the LLM. This feedback can be used to update and improve the LLM at regular intervals. For example, a system can be implemented in which users assess how useful or accurate a proposed visualization was. These assessments can then be used for fine-tuning the model.

In yet a further aspect, the technique according to the disclosure is integrated into existing imaging (e.g., MRT) software. The LLM can be hosted centrally directly in the teamplay cloud and communicate with the acquisition (e.g., MRT) fleet database and teamplay frontend. The teamplay cloud may comprise a (e.g., vendor-specific) solution for fleet analyses and data management.

The technique, according to the disclosure, permits a user-specific, interactive, dynamically expandable, and/or “on the fly” analysis of a variety of data sources for the interpretation of (e.g., MRT) scanner fleet activity. In the process, various information sources can be consolidated in a structured manner, and information can be efficiently combined and/or extracted for graphical visualization. The technique, according to the disclosure, permits an iterative investigative method of working in the analysis of fleet deviations, statistics, and trends, and/or forms of presentation.

A general challenge is posed by unstructured data from which it is difficult to infer a feedback control system for (e.g., MRT) scanner fleet optimization.

The technique, according to the disclosure, can preferably be hosted or implemented on an edge device (for short: EDGE, e.g., on the computing unit 200), which is a bidirectional connection and/or a data node to the respective imaging systems or scanners. For example, an edge hosting may be preferred for statutory data protection reasons (in particular with regard to patient data). Alternatively, a hosting or implementation of the technique according to the disclosure may be realized in a cloud.

Control loop scenarios can relate to an energy efficiency of the imaging systems or scanners, a scan efficiency of the imaging systems or scanners (in particular during a measurement or an image acquisition) and/or a utilization of the scanning capacity of the imaging systems or scanners.

An exemplary aspect of a control loop in relation to an energy efficiency of the imaging systems or scanners may comprise the requirement to determine (and/or output) the average energy consumption of the MRT systems in the past month and to determine (and/or output) which systems or scanners deviate sharply from the average, in particular upwardly, in terms of energy consumption. A further requirement may be to check whether an energy-saving mode (also: Eco Power Mode) is active on the systems or scanners. If the energy-saving mode is not active, it can be activated via a system programming interface (technical term: application programming interface, API). The LLM can generate program code for activating the energy-saving mode for some or all (e.g., N) imaging systems or scanners. In the process, a Boolean flag can be set in a service software layer of the respective scanner.

Alternatively, or in addition, a predetermined idle time (also: dead time) can be checked, after which the imaging system or scanner switches to the energy-saving mode (also: Eco Mode). The “idle time” can be reduced across the entire fleet so that the fleet switches earlier into the standby mode (also: energy-saving mode). At the same time, an integer value that defines the idle time in milliseconds (ms) can be set in the control software. The gradient amplifier (GPA) of an MRT scanner switches to the standby mode, for example, as a function of said integer value.

In a further exemplary aspect, a control loop in relation to a scan efficiency of the imaging systems or scanners, in particular during a measurement or an image acquisition, comprises that an average measured patient number per (e.g., MRT) scanner over the past month is determined (and/or output). Out of, for example, two deviating imaging systems or scanners (in particular from the average), the average scan time per body region can be determined (and/or output) and it can be determined (and/or output) which body region deviates on average from the other imaging systems or scanners. The acquisition protocol (and/or individual measurement steps) for the deviating body regions and their individual protocol measurement time can be determined (and/or output). Deviations may exist in a number of measurement steps and/or in a parameterization of the individual measurement steps. As a correction measure, a deviating measurement program or image acquisition program can be replaced by the corresponding program of another system.

According to option A, the protocol is exported by system A onto a network drive, an EDGE, and/or cloud as a protocol structure and programmatically imported by system B.

According to an option B, a protocol update ‘push’ to the system is triggered by a central protocol management server (Teamplay MR Protocol Module). A communication with the central server can be triggered via the HTTP REST Interface.

In a further exemplary aspect, a control loop in respect of a utilization of the scan capacity of the systems comprises the determination (and/or output) of a ‘table time’ portion of the scanner fleet, e.g., the percentage portion of the scanner ‘working time’, in which a patient lies on the table (also: patient couch). For the scanners with a low “table time”, a time characteristic of the past week is determined (and/or output), and “table time” ranges are marked. A percentage distribution of the measured body regions in which there are the most “table time” gaps (and/or dead time of the scanner) is determined (and/or output). For example, one result may be that head measurements always have many dead times. As a correction measure, the slot duration for brain examinations can be lowered from 40 to 25 minutes in the scheduling system via a scheduling API.

Independent of the grammatical term usage, individuals (e.g., users) with male, female, or other gender identities are included within the term.

Insofar as not already described explicitly, individual aspects or their individual aspects and features which are described with reference to the drawings can be combined with one another or interchanged without limiting or extending the scope of the described disclosure if such a combination or such an interchange is useful and in the spirit of the present disclosure. Advantages which are described in relation to a specific aspect of the present disclosure or in relation to a specific figure are, wherever this applies, also advantages of other aspects of the present disclosure.

1. A computer-implemented method for controlling or regulating an imaging medical device in a fleet of imaging medical devices of a predetermined modality, the method comprising:
accessing at least one digital database in order to receive measurement metadata with respect to at least one image acquisition or with respect to the imaging medical device, wherein the image acquisition was performed using the imaging medical device;
analyzing, using a large language model, LLM, the received measurement metadata with respect to at least one technical parameter of the image acquisition and/or of the imaging medical device;
determining a program code based on the analysis of the at least one technical parameter, wherein the program code for controlling or regulating the imaging medical device is determined; and
providing the determined program code.

2. The method as claimed in claim 1, wherein the predetermined modality is selected from a group consisting of: magnetic resonance tomography (MRT), computed tomography (CT), positron emission tomography (PET), single-photon emission computed tomography (SPECT), X-ray, and hybrid methods of the above modalities.

3. The method as claimed in claim 1, wherein the program code comprises a machine code.

4. The method as claimed in claim 1, wherein the step of providing comprises an outputting at a user interface (UI), and wherein the method further comprises:
receiving a user input via the user interface (UI) with respect to the provided program code;
modifying the determined program code based on the received user input; and
providing the modified program code.

5. The method as claimed in claim 1, further comprising:
checking the determined or modified program code with respect to at least one consistency condition,
wherein the step of providing the program code is performed when a result of the checking reveals that the at least one consistency condition has been met.

6. The method as claimed in claim 5, wherein the at least one consistency condition comprises an executability of a machine code, a compiler cross-check, a syntax analysis, a technical check, a reliability, a compliance with specified regulations and guidelines, and/or a validity.

7. The method as claimed in claim 1, further comprising:
receiving a user input via a user interface (UI) in order to determine the program code based on the received user input; and/or
post-training the LLM using the received measurement metadata and/or using the received user input.

8. The method as claimed in claim 4, wherein the user interface (UI) comprises a graphical user interface (GUI) and/or a microphone.

9. The method as claimed in claim 1, wherein the step of providing the determined or modified program code comprises an outputting of a control signal or regulation signal.

10. The method as claimed in claim 1, wherein the control signal or regulation signal is determined for:
activating an energy-saving mode;
modifying an idle time up to the activation of the energy-saving mode;
modifying or replacing an acquisition protocol of at least one imaging medical device; and/or
modifying time scheduling plans for image acquisitions.

11. The method as claimed in claim 1, wherein the at least one digital database comprises at least one vector database and/or an embedded database system.

12. The method as claimed in claim 1, wherein the analysis comprises a semantic attribution and/or structured consolidation of the measurement metadata.

13. The method as claimed in claim 1, wherein the at least one technical parameter comprises:
an energy consumption of an imaging medical device per unit time;
a number of image acquisitions by an imaging medical device per unit time;
a number of image acquisitions per unit time as a function of an acquisition protocol; and/or
a number of image acquisitions per unit time as a function of an acquired anatomical region of a patient group.

14. The method as claimed in claim 1, wherein the method is performed on a central computing unit, on an edge device, and/or in a cloud.

15. A computing unit for controlling or regulating an imaging medical device in a fleet of imaging medical devices of a predetermined modality, the computing unit comprising:
a database interface which is embodied for accessing at least one digital database in order to receive measurement metadata with respect to at least one image acquisition or with respect to the imaging medical device, wherein the image acquisition was performed using the imaging medical device;
an analysis module which is embodied for analyzing, using a large language model (LLM) the received measurement metadata with respect to at least one technical parameter of the image acquisition and/or of the imaging medical device;
a program code determination module which is embodied for determining a program code based on the analysis of the at least one technical parameter, wherein the program code is determined for controlling or regulating the imaging medical device or for analyzing measurement metadata of the imaging medical device; and
a program code interface or a program code provider module which is embodied for providing the determined program code.

16. A system for controlling or regulating an imaging medical device or for analyzing measurement metadata of an imaging medical device in a fleet of imaging medical devices of a predetermined modality, the system comprising:
a computing unit as claimed claim 15; and
at least one digital database in which measurement metadata is stored.
